(19) United States
(12) Patent Application Publication
(10) Pub. No.: US 2025/0387048 A1
(43) Pub. Date: Dec. 25, 2025

(54) BIOMETRIC SENSORS

(71) Applicant: SM 24 LTD, London (GB)

(72) Inventors: James Mayo, Paradise Valley, AZ (US); Coralie Gallis, Las Vegas, NV (US); Martyn G. Boutelle, London (GB); Sally Gowers, London (GB); Ian Stuart Hough, Maidstone Hertfordshire (GB)

(21) Appl. No.: 18/877,076

(22) PCT Filed: Jun. 21, 2023
(86) PCT No.: PCT/GB2023/051629

§ 371 Date: Dec. 19, 2024

Related U.S. Application Data
(60) Provisional application No. 63/353,933, filed on Jun. 21, 2022.

(51) Int. Cl. A61B 5/145; A61B 5/00; A61B 5/1477; A61B 5/1486

(52) U.S. Cl. CPC A61B 5/14521; A61B 5/14532; A61B 5/14546; A61B 5/1477; A61B 5/1486; A61B 5/4266; A61B 5/681; A61B 5/6833; A61B 2562/166; A61B 2562/227

(57) ABSTRACT

A sensor for measuring a biometric parameter of a bodily fluid, the sensor comprising: a sensor body (20), at least one electrode extending in the sensor body, whereby an end of the at least one electrode is exposed at an end of the sensor body, a sensor bead (30) disposed at the end of the sensor body and in contact with the exposed end of the at least one electrode.

### FIELD OF THE INVENTION

The present invention relates to a sensor for measuring a biometric parameter of a bodily fluid.

### BACKGROUND

With fitness regimes becoming ever more popular, there has been a lot of focus on personalising training. Sweat analysis in particular gives an indication of health and fitness. Much of the prior art aims to collect sweat and then subsequently analyse the collected sweat in a laboratory environment. However, the average sweat rate amounts only to approximately 1-5 nl per minute per gland. With only 100 glands per cm², this poses the problem that it is difficult to collect a sufficient amount of sweat for laboratory analysis and that sweat is not fresh by the time it is analysed under laboratory conditions, thereby reducing the accuracy of any results taken.

In an alternative approach, a sensor and integrated electronics are provided in a patch, which is adhered to the skin. Sweat or other bodily fluid is wicked from the skin through the patch to a sensor and the integrated electronics output a sensing result for, or signals that can be processed to establish measurements of, one or more parameters.

However, there are problems with wicking sufficient fluid to the sensors to provide accurate, fast (preferably real-time) results. In addition, integrating the electronics in the sensor patch is wasteful as the sensor cannot be used indefinitely.

It is therefore an object of the present invention to address at least one of the problems in the prior art.

### SUMMARY

According to a first aspect of the present invention, there is provided a sensor for measuring a biometric parameter of a bodily fluid, the sensor comprising: a sensor body; at least one electrode extending in the sensor body, whereby an end of the at least one electrode is exposed at an end of the sensor body; a sensor bead disposed at the end of the sensor body and in contact with the exposed end of the at least one electrode.

Preferably, the sensor bead is hydrophilic and is configured to equilibrate with the bodily fluid by molecular or ionic exchange with the bodily fluid; and the at least one electrode is configured to obtain a signal relating to the concentration of the hydrophilic sensor bead relating to the parameter.

Preferably, the sensor further comprises a sensor body, the at least one electrode being disposed in the sensor body.

Preferably, wherein the sensor body comprises a tube or conduit.

Preferably, a hole is formed in a wall of the tube or conduit and the at least one electrode is disposed in the hole.

Preferably, the sensor further comprises a plurality of electrodes and a plurality of holes in the sensor body, each electrode being disposed in a respective hole, whereby the electrodes are insulated from one another.

Preferably, wherein the sensor body comprises a material, for example a cured adhesive, in which the at least one electrode is embedded.

Preferably, the sensor bead comprises a hydrophilic hydrogel encapsulating an enzyme.

Preferably, wherein the hydrogel is polyethylene diglycidyl ether (PEG-DE).

Preferably, the enzyme is at least one of glucose oxidase and lactate oxidase.

Preferably, the sensor bead is coated with a diffusion limiting layer.

Preferably, the diffusion limiting layer is formed of polyurethane.

Preferably, the at least one electrode comprises a working electrode, and an exclusion layer is provided on the working electrode at the interface between the working electrode and the sensor bead.

Preferably, the exclusion layer is formed of m-phenylenediamine (mPD).

Preferably, the at least one electrode comprises a working electrode and at least one of a counter electrode and a reference electrode.

Preferably, the working electrode is formed of platinum.

Preferably, the reference electrode is formed of silver or silver chloride.

Preferably, the diameter of the at least one electrode is 10-100 μm.

According to another aspect of the present invention, there is provided a sensor patch for measuring a biometric parameter of a bodily fluid, the patch comprising: a patch body; a sensor bead; and at least one electrode in contact with sensor bead; wherein the sensor bead is exposed on a first side of the patch for contacting a bodily fluid of a user.

Preferably, the sensor patch further comprises a sensor body disposed in or on the patch body, the sensor bead being disposed at one of the sensor body the at least one electrode being disposed in the sensor body.

Preferably, the sensor body comprises a tube or conduit.

Preferably, the sensor body comprises a material, for example a cured adhesive, in which the at least one electrode is embedded.

According to another aspect of the present invention, there is provided a sensor patch for measuring a biometric parameter of a bodily fluid, the patch comprising: a patch body; and the sensor as set out above, wherein the sensor bead is exposed on a first side of the patch for contacting a bodily fluid of a user.

In the foregoing aspects, it is preferred that the sensor body extends to a second side of the patch body opposite the first side and the at least one electrode is exposed on the second side.

Preferably, the sensor patch further comprises a layer of polymer material on a second side of the patch body opposite to the first side, wherein the polymer material is harder than the material of the patch body, and the at least one electrode extends through the layer of polymer material.

Preferably, a plurality of sensor beads is exposed on the first side of the patch.

Preferably, the sensor bead protrudes from the surface of the patch on the first side whereby it can be pressed in contact with the user's skin when the patch is worn

Preferably, the sensor bead is at least partly recessed in the patch.

Preferably, the patch is configured to be held in contact with a user's skin.

Preferably, the sensor patch further comprises electronics for processing signals received from the at least one electrode.

According to another aspect of the present invention, there is provided a sensor device comprising: a base unit; and the sensor patch as set out above, wherein the sensor patch is removably attachable to the base unit, and the base unit comprises electronics for processing signals received from the at least one electrode.

According to a further aspect of the present invention, there is provided a kit for measuring a biometric parameter of a bodily fluid, the kit comprising: the sensor device as set out above; and a device configured to communicate with the base unit.

According to a further aspect of the present invention, there is provided a method of making a sensor, comprising: disposing at least one electrode in a sensor body; depositing a sensor bead on the end of the sensor body in contact with the at least one electrode.

Preferably, the sensor body comprises a tube, the method comprising: introducing an adhesive into the tube surrounding the at least one electrode; curing the adhesive; removing an end of the tube to expose an end of the at least one electrode.

Preferably, disposing at least one electrode in a sensor body comprises forming a hole in the sensor body and disposing the at least one electrode in the hole.

Preferably, the sensor body comprises a tube hole and the hole is formed in a wall of the tube.

Preferably, the method further comprises forming a plurality of said holes and disposing said electrodes in respective ones of the holes.

Preferably, the method further comprises, before depositing the sensor bead, providing an exclusion layer on a surface of an electrode.

### BRIEF DESCRIPTION OF THE DRAWINGS

Embodiments of the present invention will now be described by way of example only, with reference to the accompanying drawings, in which:

FIG. 1 is a schematic cross-section of a sensor patch according to the present invention;

FIG. 2 shows a cross-section of a sensor for use in the sensor patch;

FIG. 3 shows a cross-section of the sensor tip for FIG. 3 before a bead has been formed;

FIG. 4 is a graph representing the effects of a diffusion limiting layer on the bead;

FIG. 5 is a schematic representation of a cross-section of the sensor patch combined with a base unit to form a sensor device;

FIG. 6 is a schematic representation of a cross-section of the base unit shown in FIG. 5;

FIG. 7 is a schematic representation showing the connection of a circuit board and a metallic pad through a casing of the base unit;

FIG. 8 is a schematic representation of a cross-section of the sensor patch showing a first arrangement in which electrodes are exposed for connection to a base unit;

FIG. 9 is a schematic top view showing the first arrangement showing positions at which pads of the base unit and electrodes of the sensor patch are connected;

FIG. 10 is a schematic representation of a cross-section of the sensor patch showing a second arrangement in which pads of the base unit and electrodes of the sensor patch are connected;

FIG. 11 is a schematic representation of a cross-section of the sensor patch showing a third arrangement in which pads of the base unit and electrodes of the sensor patch are connected;

FIG. 12 is a schematic top view showing positions at which pads of the base unit and electrodes of the sensor patch are connected in the second or third arrangement;

FIG. 13 is a schematic representation in which multiple sensors are provided in a sensor patch;

FIG. 14 is an illustrative cross-section of another embodiment of a sensor;

FIG. 15 is schematic drawing showing how the sensor patch and the base unit can be joined;

FIG. 16 is a schematic drawing showing an alternative way in which the sensor patch and the base unit can be joined;

FIG. 17 is a schematic representation of a bottom view of smart device incorporating the sensor device in a wrist band;

FIG. 18 is a schematic representation of a side view of the smart device in FIG. 17; and

FIG. 19 is a schematic representation of a casing for the base unit and one or more sensor patches.

### DESCRIPTION OF THE INVENTION(S)

The drawings are not to scale. Features may be shown expanded relative to other features, or with aspect ratios adjusted, in order to aid illustration. Likewise, features may be omitted from the drawings for ease of understanding.

In the present invention, there are provided a sensor (sensor) 50, a sensor patch 100 incorporating at least one sensor 50 and a sensing device 1 incorporating a patch 100.

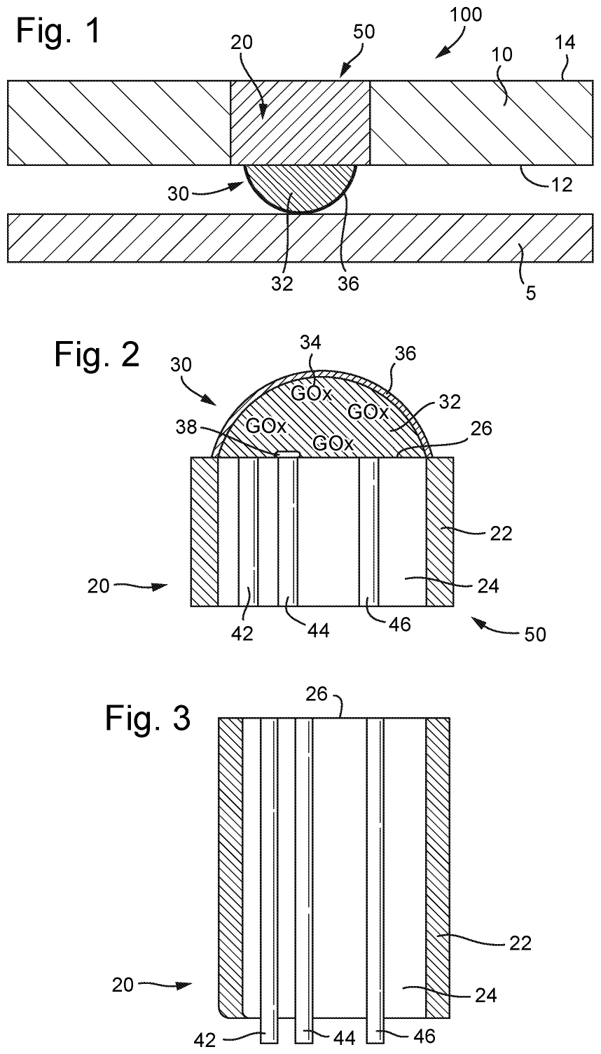

An embodiment of a sensor patch 100 is shown FIG. 1. In this embodiment, the patch 100 comprises a patch body 10 with a sensor 50 disposed in it. The sensor 50 comprises a sensor body 20 with any suitable cross-sectional shape and area. It preferably extends through the patch 100. A hemispherical, domed or similarly-shaped sensor bead 30 is provided at the end of the sensor body 20 so that the sensor bead 30 extends out of the patch or at least is not covered by patch material. In this way, the bead 30 is positioned in direct contact with or close to the skin when the patch 100 is adhered to the skin 5 of a user, as schematically shown in FIG. 1 so that the bead 30 contacts sweat from the skin. Although not shown in FIG. 1, at least one electrode extends at least part way through the sensor body 20 and terminates in contact with the sensor bead 30.

In practice, when sweat comes into contact with the sensor bead 30, the sweat and the sensor bead 30 undergo a molecular or ionic exchange, preferably so as to reach a charge equilibrium. A signal corresponding to any changes in the charge or composition of the sensor bead 30 is generated in the electrode, and this signal can be used to determine the composition of the sweat that has interacted with the sensor bead 30, thereby providing biometric data in real-time.

The sensor 50 may otherwise take any appropriate form. Because the sensor bead 30 is exposed on the bottom surface of the patch 100 and is held in contact with or close to the user's skin 5 when the patch is applied, the user's sweat comes into contact with the sensor bead 30 as soon as it is produced. This allows the sensor 50 to have an extremely rapid response time, and improves the capability of measuring parameters in real time. By contrast, prior art sensor patches may involve a complicated wicking structure to wick sweat past the sensing area. In such prior art patches, there is therefore a delay between production of sweat and sensing. Moreover, the composition of the sweat may be affected by wicking, and it is necessary for a relatively large amount of sweat to be produced before it can reach the sensing area. These issues are all addressed by the present invention. Moreover, as discussed below, by providing a small, self-contained sensor bead of a domed or similar shape that comes into direct contact with sweat at the place of production, it is possible to further reduce response times.

The sensor bead 30 is preferably hydrophilic and more preferably comprises a hydrogel 32. The sensor bead 30 is preferably covered by a polyurethane (or parylene or other polymer) diffusion limiting layer 36, which restricts the substrate entry to the hydrogel 32. A plurality of such beads 30 can be provided in a single patch 100.

The sensor bead 30 is small in volume and preferably highly curved, taking on an approximate hemispherical or otherwise domed shape so as to minimise the signal to noise ratio of the resulting data. However, flatter beads are also possible and within the scope of the invention. The radius of curvature (or maximum radius of curvature in cases where a cross-section of the bead is not circular) of the sensor bead is preferably in the range of—μm-1 mm, and more preferably 30-100 μm. The volume of the sensor bead is preferably in the range of 0.5 fl-10 μl. This increases the flux of molecular or ionic exchange at the surfaces of the hydrophilic sensor bead 30, thereby increasing the rate of charge or molecular equilibration. The flux of molecular or ionic exchange is also increased at the surface of electrodes that terminate in the sensor bead 30, thereby increasing the speed of response of the electrode to concentration or composition changes. Accordingly, the rate of detection of any changes in charge is increased, providing a rapid response time for accurate real-time results.

The sensor 50 may be an amperometric sensor comprising two or three electrodes embedded within or otherwise exposed to the hydrophilic sensor bead 30. The sensor 50 could optionally work by a coulometric method, in which the charge concentration of a specified variable, e.g. a biomarker molecule, in a given hydrophilic sensor bead 50 is monitored by measuring the integrated current. Specifically, the material (for example, hydrogel 32) in each sensor bead 30 has a predetermined volume and chemical composition. Any change in the concentration of a given variable indicates any molecular or ion exchange that has taken place.

The coulometric method has the benefit of making it unnecessary to perform calibration techniques. This is because the total amount of charge obtained will be the same, irrespective of the detection efficiency of a given sensor 50.

Preferably, the sensor 50 works by an amperometric method in which the current is measured as a measure of concentration, thereby indicating the amount of the analyte in the sweat or other bodily fluid.

The amperometric sensors 50 can determine tissue metabolism by measuring lactate levels which are an indicator of global and local muscle fatigue and recovery. The skin patch may also monitor glucose levels as a non-invasive means of measuring blood glucose. Other indicators may also be measured, for example cortisol to monitor stress levels.

Polymers 32 are provided to bind the recognition species for the analytes to be measured e.g. lactate and/or glucose. In some embodiments, recognition of targeted species is performed by biorecognition moieties such as a bioprotein which has a specific binding site for the analyte. Examples include but are not limited to: enzymes, antibodies, membrane channel proteins or binding molecules, such as valinomycin (for K+ ions). In other embodiments, recognition is achieved by use of synthetic binding sites chosen to bind an analyte selectively compared to other chemical species. Examples include but are not limited to: aptamers and synthetic ionophores.

The polymers are predetermined according to the species to be measured. The polymers may be provided on the sensor electrodes.

As noted above, the polymer is preferably a hydrogel 32, which is a highly hydrophilic polymer that incorporates a high-water content. In an amperometric sensor 50, the binding sites are chemically joined to this polymer to keep them within the bead 30. An example of a hydrogel 32 is a hydrogel comprising 30 mg/ml albumin, 60 mg/ml PEG-DE, 2% glycerol in 0.01M PBS but other forms of hydrogel may be used and tuned to give desired properties. The hydrogel 32 provides a continuously conductive environment that allows the beads 30 to provide electrical conductivity at all times.

Furthermore, the amperometric sensor 50 can be switched on and off. Switching on the electrodes to make a circuit depletes the charge, and switching off the circuit allows for greater equilibration with sweat that passes the sensor bead 30 in cases where the rate of equilibration is slow. It is therefore preferable to periodically switch on and off the sensor 50, which allows smaller concentrations of measured ions in the sweat to be detected. In addition, the sensitivity of the amperometric sensor 50 to smaller volumes of sweat can be improved. Alternatively, it is possible to provide continuous detection by keeping the electrodes on.

The electrodes may comprise gold or carbon, while the surfaces of the electrodes are platinum-based. As discussed below, it is preferred that the electrodes are platinum and silver. The electrodes can have a diameter of between 10 and 50 μm. Furthermore, connectors together with the accompanying interconnective strings connecting each sensor are provided as wires having diameters of between 10 and 50 μm.

The invention however is not limited to amperometric sensors 50. In other embodiments, the sensor 50 is a potentiometric sensor comprising a first indicator electrode embedded within the hydrophilic sensor bead 30 and a second reference electrode external to the hydrophilic sensor bead 30 that measures the difference in voltage between the first and second electrodes. The potentiometric sensor monitors the total potential change of a specified variable, e.g. potassium, sodium and/or chloride ions, in a given hydrophilic sensor bead 30, which indicates any ion exchange that has taken place. Such potentiometric sensors 50 can be used to detect hydration levels.

In an embodiment, one or more reference sensors 50 are provided in the skin patch such that they are not exposed to sweat. As such, any variation in the charge concentration is dependent on body temperature, but not on sweat. The data from the reference sensor(s) 50 can then be compared to sensors 50 exposed to sweat in order to perform temperature calibration. This is particularly beneficial when used during exercise as a means of compensating for any changes in skin temperature that may affect the data obtained.

In an embodiment, the sensor body 20 is not embedded in the patch 100, but is instead disposed on the bottom 12 of the patch 100. It will be apparent that the sensor body 20 can be very short so the sensor 50 need not protrude to far from the bottom surface 12 of the patch 100 in such an arrangement. The electrode wires 42, 44, 46 may then extend out of the sensor body 20 and through the patch body 10, or even along the bottom of the patch body 10 for at least a short distance.

Alternatively, only part of the sensor body 20 is embedded in the patch body 10.

The skin patch 100 of the first embodiment optionally comprises an equilibration rate means 36 for controlling the rate of equilibration between the sensor bead 30 and sweat that contacts the sensor bead 30. This equilibration rate means takes the form of a diffusion limiting layer or membrane 36 that coats the sensor bead, as shown in FIG. 1. The diffusion limiting layer 36 may include a polymer film with a lower water content than the hydrogel 32 or other bead material. In some embodiments, the diffusion limiting layer 36 may be a substantially dense polymer film with nanopores distributed through the film.

In practice, the diffusion limiting layer 36 loads molecules external to the sensor bead 30 internally into the hydrogel or other material core 32 by a suitable mechanism. For example, in some embodiments, the diffusion limiting layer 36 provides a passive conduit to the internal hydrogel 32 of the sensor bead 30.

In other embodiments, the diffusion limiting layer 36 is selectively permeable and only allows predetermined ions or molecules to pass by preferentially facilitated diffusion of species along a concentration gradient. In this way, the sensor beads in different sensors 50 (whether in the same patch 100 or different patches 100) can be coated with different diffusion limiting layers 36 in order to sense different components in the sweat. This means that desired analytes may be selected to permeate into the sensor bead 30, while other molecules present in sweat that might give an interfering response can be prevented from permeating into the sensor bead 30.

The diffusion limiting layer 36 may also act as a protective layer having one or both of two functions. The first is improving the structural integrity of the sensor bead 30 so that it is less prone to damage from contact with outside bodies (including the user's skin). The second is keeping contaminants from the outside from entering the sensor bead 30. Such contaminants may be germs, viruses and other foreign bodies. In this way, the diffusion limiting layer 36 acts to prevent biofouling of the sensor bead 36.

It will be apparent that a separate protective layer 36 may be provided in addition to or instead of the diffusion limiting layer 36. In addition, the diffusion limiting layer 36 may be comprised of several different layers, which may selectively limit diffusion of different (or the same) ions, molecules or other bodies into the sensor bead 30.

FIGS. 2 shows a preferred configuration of a sensor 50 according to the invention. Such a sensor 50 is preferably used in the patch 100 in FIG. 1 and the patches 100 and sensor devices 1 described and/or illustrated throughout this specification. It will be appreciated that the patches 100 and sensor devices 1 may also use other sensors. FIG. 3 illustrates a preferred step in its manufacture.

As shown in FIG. 2, the sensor 50 comprises a thin, inert and flexible tube 22 forming the outside of the sensor body 20. The tube may be made of PEEK, PVC, polycarbonate or polyurethane (PU), for example and preferably has an external diameter of 100 μm-1 mm.

Electrode wires 42, 44, 46 are pushed through the empty tube 22 as shown in FIG. 3. The electrode wires 42, 44, 46 can comprise a working electrode 42, a counter electrode 44 and a reference electrode 46. However, since the currents involved are very small, it is also possible to use only two electrodes. Thus, depending on the application and/or specific configuration, the counter electrode 44 can be removed. This improves ease of manufacture and reduces cost. The sensor may also have only one electrode 42. Alternatively, additional electrodes can be added to improve sensitivity/accuracy and/or provide redundancy. Preferably, the working and counter electrode wires 42, 44 are platinum wires and the reference electrode wire 46 is an Ag wire, which is preferably coated with AgCl. The electrode wires 42, 44, 46 preferably have an external diameter of 10-100 μm. It is preferred that the electrode wires, especially the working and counter electrode wires are isonel-insulated. However, the materials of the sensor body may suffice to insulate the electrode wires so that the wires themselves are not provided with an insulating coating.

In one embodiment, there are
Two Pt wires, each of total diameter 24 μm including insulation of thickness 2 μm.
One Ag wire of total diameter 35 μm including insulation of thickness 5 μm
PEEK tubing of 360 μm outer diameter and any of 100 μm, 125 μm and 150 μm internal diameter

In a preferred embodiment, there are.
Two Pt wires, each of total diameter 75 μm including insulation of thickness 12.5 μm.
One Ag wire of total diameter 65 μm including insulation of thickness 7.5 μm
Portex® PVC tubing of 800 μm outer diameter and 400 μm internal diameter

The tube 22 is then filled with a curable adhesive 24, for example an epoxy adhesive or a UV curable adhesive, to surround the electrodes 42, 44, 46. After curing, one end of the filled tube 22 is cut to provide a flat surface 26 with ends of the electrode wires 42, 44, 46 being exposed. The electrode wires 42, 44, 46 protrude from the other end of the tube 22 for connection to an electronic circuit for signal processing.

An exclusion layer 38 is then preferably deposited on the exposed surface of the working electrode 42 to improve the selectivity of the electrode 42. The exclusion layer 38 is preferably a layer of polymer material, preferably m-phenylenediamine (mPD)—see FIG. 2. This completes formation of the sensor body.

A hydrogel core 32 is then formed on the cut, flat surface 26 over the exposed ends of the electrode wires 42, 44, 46. The hydrogel core 32 encapsulates an enzyme 34 for reacting with the substrate of choice. In the example shown, the encapsulated enzyme 34 is GOx (glucose oxidase) for glucose. Alternatively, LOx (lactose oxidase) for lactate may be encapsulated. As discussed above, enzymes or enzyme systems or other molecular recognition systems (for example using antibodies and aptamers) for other substrates, such as cortisol, may be included instead. Preferably, the hydrogel core 32 is polyethylene glycol diglycidyl ether (PEG-DE) hydrogel. The hydrogel core 32 and exclusion layer 38 may also be adjusted for sensing of other analytes.

The hydrogel core 32 may be formed by deposition or dipping, for example. The hydrogel core 32 forms a substantially hemispherical surface, which is not limited to an exact hemisphere but includes other domed shapes. The degree of the doming can be controlled by adjusting the diameter of the hydrogel core 32 parallel to the cut surface 26 of the sensor body 20, and by ensuring a relatively high contact angle between the hydrogel/enzyme mixture and the flat surface 26. An appropriate contact angle can be achieved by selecting appropriate materials for the tube 22 and/or the cured adhesive 24 relative to the hydrogel 32.

After application to the end of the filled tube 22, the bead of hydrogel/enzyme mixture 32 is cured and dipped into a polyurethane polymer (PU) to form an outer shell 36. The PU shell 36 acts as a diffusion limiting layer. It will be appreciated that the diffusion limiting layer 36 can be formed with other materials and using a different process. This completes formation of the sensor bead 30, and of the sensor 50 as a whole.

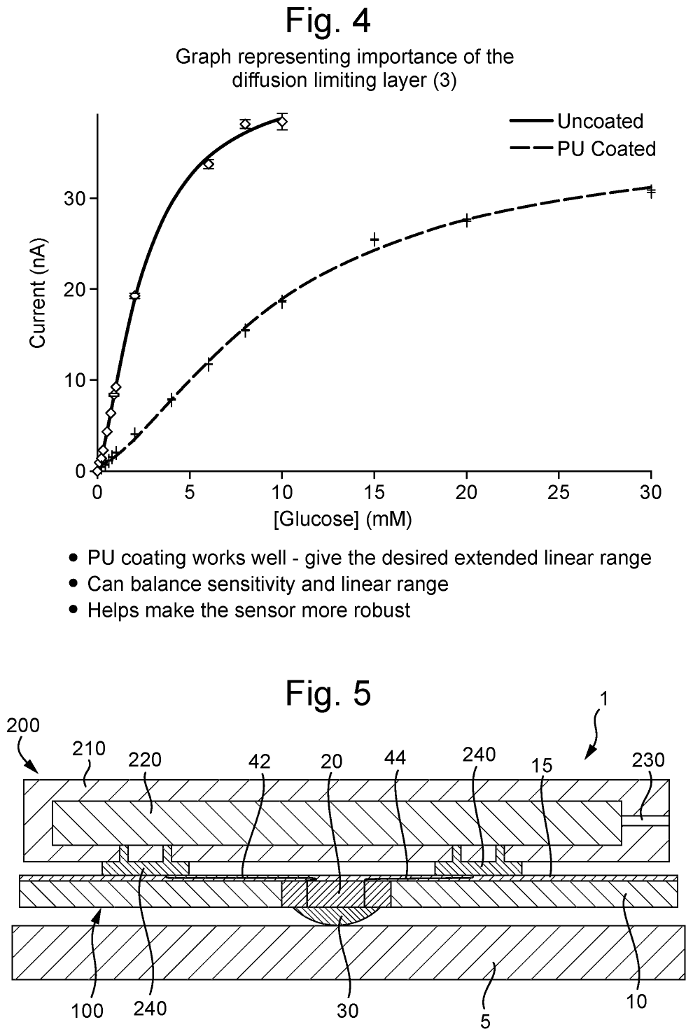

Although not essential, the diffusion limiting layer 36 has the significant advantage that it limits the amount of the substrate that passes through from the sweat surrounding the bead 30 into the cured hydrogel/enzyme mixture 32. As shown in FIG. 4, by limiting the amount of diffusion of the substrate, in this case glucose, into the bead, is possible to achieve a more linear response of current to concentration of substrate for a wider range, improving accuracy of sensing. In addition, by controlling the amount of substrate in the bead 30, the rate of use of the enzyme is reduced and the sensor 50 can last longer. Finally, the diffusion limiting layer 36 makes the sensor 50 more robust.

The sensor described above may last several weeks or even months in use, and can be stored for long periods in a freezer and/or sealed in a pouch before use.

Where the diffusion limiting layer 36 is not provided, the cured hydrogel 32 forms the sensor bead 30.

As shown in FIG. 1, the sensor body 20 can be embedded in a patch 100 so that the sensor bead 30 is exposed on one side and the electrode wires 42, 44 (not shown in FIG. 1) extend out of the adhesive-filled tube 22 on the other side. The main body 10 of the patch 100 is preferably formed of PDMS or a like material, which can be adhered to a user's skin. The main body 10 of the patch preferably provides compliance with the shape of the user's skin where the patch 100 is applied and preferably also provides comfort. It is not necessary for the patch 100 to adhere to the user's skin 5 and it could be held in place with a band or the like.

The sensor bead 30 preferably protrudes from the bottom surface 12 of the patch 100 so that it is pressed in contact with the user's skin 5 when the patch is worn 100. However, the sensor bead 30 may be partly or fully recessed in the patch 100, so long as it is held in contact with or sufficiently close to the user's skin 5 when the patch 100 is worn.

In prior art designs of sensor patches, it has been necessary to wick sweat from a user's skin to a sensing area to achieve sufficient amounts or concentrations of analytes for sensing. By contrast, in the present invention, there is no need to provide any means of wicking sweat or other fluids from the user to the sensor bead 30. It has been found that accurate results can be achieved without wicking sweat to the sensor bead 30, even when the user is at rest, due to the high sensitivity of the sensor bead 30—that is, even without wicking of sweat to the bead, there is no need for the user to exert him or herself to generate sufficient sweat for the sensor 50 to work accurately. In this way the sensor can be used not only for tracking glucose and other levels during exercise but also in normal day-to-day use. As such, a patch 100 incorporating the sensor 50 described herein can have a very simple construction compared to the prior art.

The patch 100 may include some means of wicking sweat away or otherwise allowing sweat to escape from the patch, but this is not essential to the working of the invention.

The patch 100 may also include one or more small heaters (not shown), preferably disposed around or near the sensor bead 30. In cases where there is little or no sweat, for example where the ambient temperature is low or the user is not moving, the heater(s) can cause the user to sweat sufficiently for the sensor 50 to function better. The heater(s) may comprise a heater electrode or wire formed of a material that heats up when a current is passed through it, as is known in the art.

The heater(s) may be powered by a power source (e.g. battery) provided on the patch 100 or the base unit 200.

The heater(s) may be controllable by the user or may be controlled automatically, for example based on any one or more of ambient temperature, ambient humidity, sensed movement of the user and functionality of the senor 50. With respect to the latter, if for example it is determined that the user is not sweating, for example because a low or zero level of analytes is detected or there has been no change to detected sweat for a prolonged period, then the heater may be switched on.

Such control can be implemented, for example, by the base unit 200 or by electronics provided directly on the patch 100. Temperature, humidity, movement etc may be determined by an external device, for example by a smart phone with which the sensor device 1 communicates, and transmitted to the sensor device 1, which may then control the heater(s) based on the received signals. Alternatively, control signals to switch on/off the heater(s) may be transmitted to the sensor device 1. Alternatively, the heater may always be on.

By providing the preferred sensor 50 described above, it is possible to provide a highly accurate sensor that requires only very small volumes of fluid, for example 2-5 nanolitres of sweat, to give accurate readings. Moreover, the sensor 50 can be easily and cheaply made using simple manufacturing techniques, and is highly robust.

It will be apparent that, although not preferred, additional layers may be provided on the upper and/or lower surfaces of the patch body 10. However, this is not a problem if the sensor bead 30 is exposed and there is some means of external connection to the electrode(s) 42, 44, 46 for the extraction of signals.

It may even be possible to cover the sensor bead 30 to the extent that sweat can be efficiently transported to the sensor bead 30. The construction of the sensor 50 shown in FIG. 2 has significant advantages, for example in terms of ease of manufacture, ease of deployment, ease of incorporation in patches and other sensor devices, sensitivity, reaction speed and robustness that remain even if the sensor bead 30 is covered and sweat is wicked or otherwise transported to the sensor bead 30.

As shown in FIGS. 2 and 3 and subsequent figures, one or more electrodes 42, 44, 46 are exposed in the sensor bead 30 and run through the tube 22. The opposite end of the electrode wire (or line, thread or filament) 42, 44, 46 is connected to, or can connect to, an electronics circuit for processing signals from the electrodes 42, 44, 46. Only the very tip of any or the electrodes 42, 44, 46 need be exposed to the sensor bead 30, or the electrode 42, 44, 46 may extend properly into the sensor bead 30.

By constructing the patch 100 as shown in FIG. 1, with the bead(s) 30 in direct contact with the skin 5, or very close to the skin 5, the sensor 50 becomes highly responsive even when only small amounts of fluid such as sweat are present, leading to fast, accurate results that can tracked in real time as the user exercises or otherwise moves. The sensor 50 is also sufficiently sensitive and responsive that accurate measurements can be taken and tracked when the user just sits or is otherwise stationary as the amount of sweat required for a reading is minimal (2-5 nl).

It will be apparent that the sensor 50 can be provided separately from the patch 100, either for subsequent incorporation in a patch 100 or for use without a patch.

Features of the preferred sensor 50 arrangement include the provision of one or more electrode wires 42, 44, 46 in a sensor body 20. This makes it easier to guide the wires 42, 44, 46 from the sensor bead 30 on the surface of the skin 5 for connection to electronics 220 used to process signals from the electrodes 42, 44, 46. The sensor body 20 also supports the sensor bead 30 comprising the hydrogel/enzyme mixture 32.

Preferably, the sensor body 20 comprises a tube 22, which may be filled with an adhesive or other resin 24. However, the tube 24 is not essential and the wires 42, 44, 46 can be held in position using only the cured adhesive encasing the wires 42, 44, 46 instead, the cured adhesive 24 also supporting the bead 30. The adhesive 24 can be applied using another method than the tube 22. Alternatively, after curing of the adhesive 22, the tube 22 can be removed.

It will be appreciated that the sensor body 20 may be formed in any other suitable way using any suitable materials. For example, it is not necessary to use an adhesive and the sensor body 20 may be formed of any suitable plastic, resin or other material. The electrode wires 42, 44, 46 may be moulded or otherwise encased in plastic or other material to form the sensor body 20 before the sensor bead 30 is formed. The cross-section of the sensor body (including the tube 22 where provided) may be circular, square, rectangular or any other appropriate shape.

In a similar way, it is possible to provide the tube without the adhesive.

In addition, although it is preferred to include at least one of the tube 22 and the adhesive 24, and further preferred to include both, it is also possible to omit both the tube 22 and the adhesive 24 or other body surrounding the wires 42, 44, 46. Thus, it is conceivable for the wires 42, 44, 46 to extend from the sensor bead 30 unsupported. In the case, the sensor bead 30 may then be held in place using the mechanical properties of the wire(s) 42, 44, 46. In a further alternative, either or both the wire(s) 42, 44, 46 and the sensor bead 30 may be supported directly by the patch body 10, e.g. the PDMS material.

In general terms, it is preferred to include some means for supporting both the sensor bead 30 and the wires 42, 44, 46, or at least one of them.

Again, although preferred, the diffusion limiting layer 36 is not an essential feature.

As explained above, the sensor bead 30 preferably has a hemispherical shape. In the present application, unless the context requires otherwise, the term ‘hemispherical’ is not limited to geometrical hemispheres and comprises segments of hemispheres and other domed shapes, including domes with partially flat tops or domes where the curvature at the top is less than the curvature at the sides. However, shapes more accurately approximating a geometrical hemisphere or a segment thereof are preferred to maximise the surface area for diffusion into the hydrogel 32/enzyme 34 mixture, as well as to maximise the amount or volume of hydrogel 32/enzyme 34 mixture per electrode, whilst at the same time providing an easily manufactured, accurate and responsive sensor 50.

Although it is preferred that the surface of the electrodes 42, 44, 46 is flush with the flat surface 26 formed by the cut tube 22 and adhesive 24, it is possible for the electrodes 42, 44, 46 to extend into sensor bead 30, including towards the middle of the bead 30.

It will be apparent that the various manufacturing steps described above can be omitted and that the order of some steps may be changed.

### Separable Patch and Base Unit

Although not shown in FIG. 1, the sensor patch 100 may be integrally provided together with electronics used to process signals from the electrodes. Specifically, the sensor patch 100 and the electronics can be integrated into the same body—that is, the electronics can be provided in or on the patch as a unit.

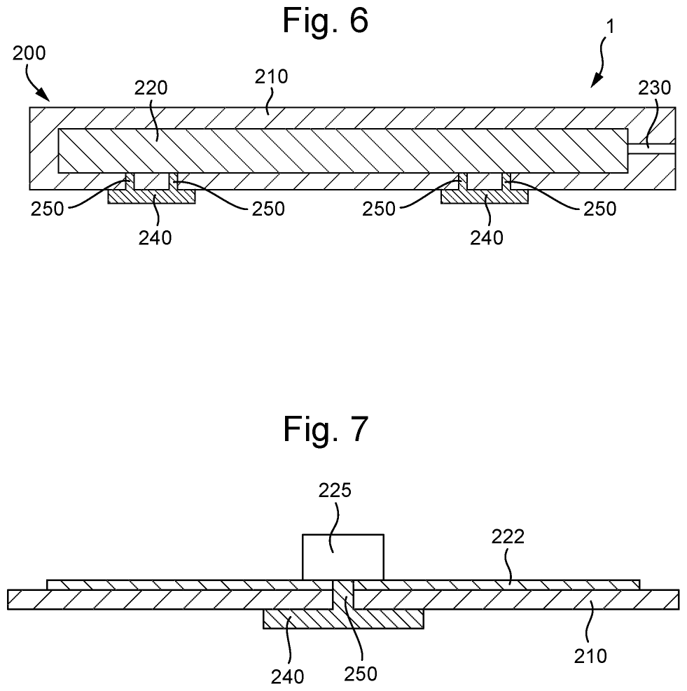

However, in another preferred concept of the invention, as shown in FIG. 5 and discussed in more detail below, the patch 100 comprising the sensor 50 is provided as a consumable, separate from a base unit 200 comprising the electronics 220. In this way, the consumable patch 100 can be attached to and removed from the base unit 200, which is shown separately in FIG. 6. When the patch 100 and the base unit 200 are attached to one another, they can be said to form a sensor device 1.

By providing a base unit 200 and a separate consumable patch 100, waste is significantly reduced and the same base unit 200 can be used with multiple patches 100. As the sensing capabilities of a patch 100 degrade, the patch 100 can be removed from the base unit 200 and swapped for another. The base unit 200 provides electronics 220 for at least partial processing of signals from the sensor 50. The base unit 200 includes means for transferring the output of processing to any suitable external device (not shown) such as a laptop, smartphone or other computing device. The external device may be or act as a gateway, for example to an application or network. Such means may comprise wired means, such as a USB-C connector 230, or wireless means, such as Bluetooth, BLE, Zigbee, near field communication (NFC), WiFi, 5G or any other suitable means of communication. The base unit 200 is provided with a charging connector 230, which may be one of USB-C, magnetic or another kind of connector, as is well-known in the art. Alternatively or in addition, the base unit 200 includes a display (not shown) for displaying to the user an output based on the signals from the sensor 200. Such a display may be provided on the external device as well or instead, for example using an app.

FIG. 5 shows the base unit 200 when separated from the patch 100. FIG. 6 shows another schematic view of components of a base unit of the invention.

The provision of a base unit 200 and a separate patch 100 presents a number of challenges.
The size of the sensor 50 is so small that a specific connector needs to be developed to connect it to the base unit 200. The thinness of the patch 100 requires to develop a connector that is thin enough and easy to connect without the risk of disconnection. The base unit 200 and the sensor 50 should be seamlessly and easily connected to and disconnected from one another.
Separate patches and base units have been previously provided in which the base unit is connected to the patch with a flexible connector. However, the skin patch needs to be as thin as possible and the flexible connector is cumbersome and difficult to maneuver. Accordingly, it would be preferable to remove this flexible connector and this is the focus of further concepts of the present invention.
However, the flexible connector has the advantage that there is no need for precise alignment between base unit and sensor patch. Once the flexible connector is removed, the sensor patch 100 needs to be aligned with the base unit 200 to ensure that the sensor electrodes 42, 44, 46 properly align with corresponding electrodes on the base unit 200.
The size of the electrodes 42, 44, 46 is so small (10-100 μm), that the connection between one electrode 42, 44, 46 from the sensor 50 onto one connector of the base unit 200 becomes very difficult. Specifically, it is difficult for a user to attach the patch 100 and base unit 200 together with sufficiently precise alignment between them, if both connectors are so small.
To minimize the impact on the environment and the cost for the user, the base unit 200 is reusable while the sensor patch 100, which is environment friendly, is a disposable component of the sensor device 1.

To address the issues described above, the present invention provides several possible solutions. In the general implementation, the following applies, although individual points are not essential:
The sensor device 1 is composed of two elements: a base unit 200 including the electronics/digital component 220 and a sensor patch 100 composed of at least one sensor 50 and a material (patch body 10) that holds the sensor (a PDMS holder for instance).
As shown in FIG. 6, the base unit 200 includes a sealed casing/packaging 210.
As previously discussed, the sensor 50 may be composed of a tube 22 and one or more electrodes 42, 44, 46.
As shown in FIGS. 5 and 6, flat metallic contact pads 240 (depending on the number and type of sensors 50) are provided on the base unit 200.
The metallic pads 240 connect the electronics component 220 and the electrodes 42, 44, 46 of the sensor 30 and make the connection from the base unit 200 to the sensor(s) patch 100.
The metallic pads 240 are linked to the electronics 220 through vertical and metallic vias 250 through the casing 210 (see FIG. 7).
The patch 100 and the base unit 200 may be joined using an adhesive (not shown). The adhesive may be provided on the base unit 200 only, the patch 100 only or both. Different adhesive materials may be provided on the base unit and patch which, when combined, form a sufficiently strong adhesive. The adhesive is preferably not applied to the electrodes 42, 44, 46 or the pads 240 or, if it is, the adhesive is electrically conductive and more preferably anisotropically conductive.
The patch 100 and the base unit 200 may also be joined in other ways, for example with a magnetic element on each, or the patch 100 may be clipped in the base unit 200 (or vice versa) as discussed below, or the patch 100 may push and twist into the base unit 200 in a screw motion.
The sensor tube 22 is held within a suitable structure, for example a polymer structure, and preferably a PDMS structure.
The PDMS (part of the sensor patch 100), if used, is a material sticky enough to hold to the base unit 200. The PDMS could have a layer such parylene that is stiff.

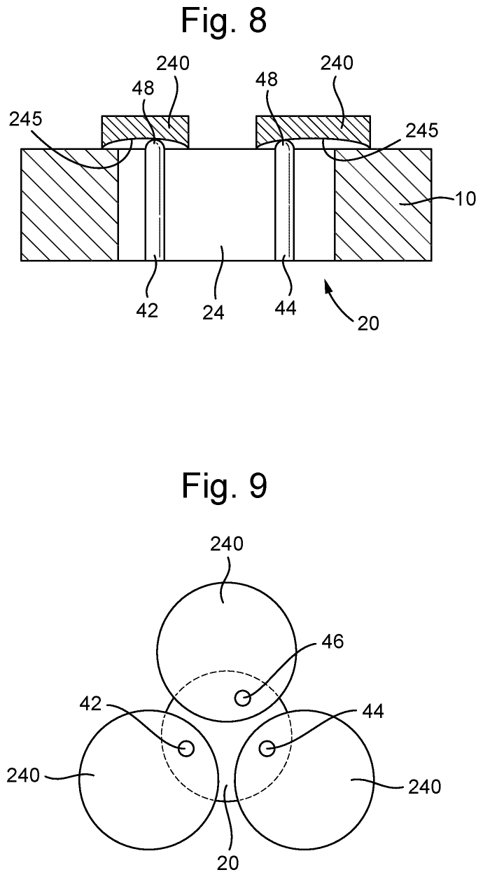

In one arrangement, the metallics pads 240 on the base unit 200 (which are, for example, ovoid or polygonal in shape in plan view) include a rounded groove or hollow shape or a cavity 245, as shown in FIG. 8. The electrodes 42, 44, 46 on the sensor 50 side include or terminate in metallic bumps or other projections 48: these make the positioning of the sensors patch easy to connect. Preferably, one or more of the following applies:
The pits or holes 245 of the pads 240 block the sensor patch 100 at the right position and avoid the patch 100 sliding on or from the base unit 200.
The concavity 245 of the connectors/metallic pads 240 on the base unit 200 allow for a good connection from sensor patch 100 to base unit 200.
The electrodes 42, 44, 46 terminate in a bump 48, a bulge or other protrusion protruding just out of the tube.

FIG. 8 shows how the protruding electrodes 42, 44, 46 contact the concavities 245 in respective pads 240. In one embodiment, fitting the patch 100 and the base unit 200 may cause the protruding electrodes 42, 44, 46 (especially if they are bump electrodes) to be crushed or otherwise deform to conform with the shape of (at least part of) the respective concavities 245 to ensure a close fit and good connection. Optionally, the lower surface of the base unit 200 may be adhered to the upper surface of the patch 100.

FIG. 9 is a schematic top view showing the base unit pads 240 as round in plan view aligned with electrodes 42, 44, 46 from the sensor body 22. The pads 240 may take any suitable shape. Other aspects of the patch 100 and base unit 200 are omitted in FIG. 9.

Semiconductor industry technology may be used to implement this solution, for example to form bump electrodes.

It is noted that the sensor tube 22 is omitted from FIG. 8 and the electrodes 42, 44 are provided in sensor body 20 without at surrounding tube. Such an arrangement could also be provided in all other embodiments. Conversely, a sensor 50 incorporating a sensor tube 22 could be used in FIG. 8

In another connection arrangement, it is preferred that one or more of the following applies:
On the side of the sensor 50, the electrodes 42, 44, 46 coming out of the tube 22 (length of 1-10 mm) are spread evenly and at a known position with a specific accuracy.
The sensor body 20 in which the electrodes 42, 44, 46 are positioned is held in a patch body 10 of one or more materials (PDMS for instance). That is, the sensor body 20 is integrated in the one or more materials.
The electrodes 42, 44, 46 once out of the sensor body 20 (sensor tube 22, if provided), are integrated in a protective layer 15 formed, for example, of parylene or PDMS or Kapton or one or more similar materials. The protective layer 15 preferably has a harder surface than the material(s) of which the body 10 of the patch 100 is made and which surround(s) the sensor body 20.
The connection between the base unit 200 and the sensor patch 100 is preferably waterproofed. Therefore e.g. parylene could be used (FDA approved) for the protective layer 15. Other hydrophobic materials could also be used to provide waterproofing. By providing a hydrophobic material, it is possible to prevent or reduce the sweat/water from getting to the electrodes 42, 44, 46 to avoid a short circuit. The upper side of the sensor 50 should preferably not come in contact with the sweat, hence the use of a hydrophobic material. As with other materials used to form the patch, it is preferred that protective layer is biocompatible, inert and non-toxic.
To facilitate the connection between the electrodes 42, 44, 46 from the sensor 50, the metallic pads 240 from the base unit 200 are at a known position. The pads 240 are large enough (from 100um to 2 mm) to allow to enable easily the connection from the electrodes to the base unit.
The contacts between the sensor/electrode to the base unit should be low resistance to let the current pass.
The contacts within the sensor patch 100 and within the base unit 200 should be sufficiently high resistance, to avoid leakage from one to another.
By way of example, a 1V amplifier in the base unit 200 might use resistance 109 ohm and require a resistance of 1011 ohms.
All the various sensors 50 (if there is more than one) in the patch 100 are at a known position. The metallic pads 240 on the base unit 200 are also at a known position.

The patch 100 and the base unit 200 may be held with adhesive or within a holder provided in the base unit casing 210 or using a screw type connector or a clip type connector.

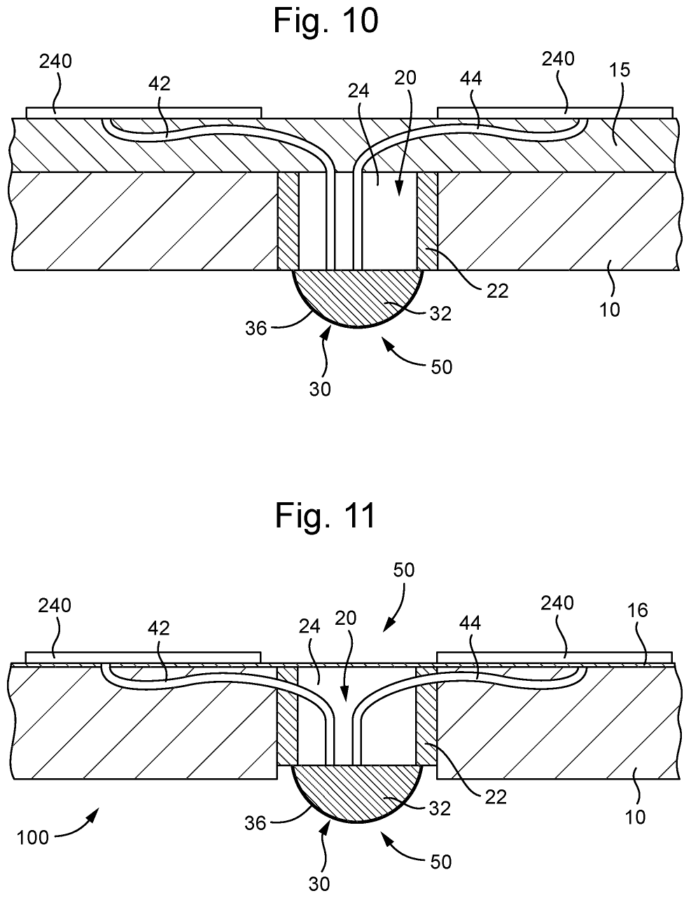

FIG. 10 schematically illustrates such an arrangement. It shows the patch 100 and the pads 240 of the base unit 200 in cross-section, but omits other elements of the base unit 200 for clarity. The electrodes 42, 44, 46 extend out from the top of the sensor tube 22, for example as filaments, and are encased in the protective layer 15 of harder material e.g. polymer material. The harder material is advantageous at least two reasons: it provides protection of the filament/electrode 42, 44, 46 and improves the mechanical property of the patch 100 itself making it easier to handle without breaking the thin filament (10-100 um). The electrode wires 42, 44, 46 terminate at the upper surface of the polymer material 15 so they are exposed. The base unit 200 is pressed against the patch 100 so that the metallic pads 240 on the base unit 200 contact the exposed surface of the electrodes 42, 44, 46. The surface area of the exposed portion of an electrode 42, 44, 46 can be controlled as desired, for example by widening the end or by running in a spiral, winding or other shape so that an extended portion of the side of the electrode wire 42, 44, 46 is exposed. By setting the electrodes 42, 44, 46 in the layer of harder material 15, the position of the exposed portion can be accurately controlled. Similarly, by using a large pad 240, it is possible to ensure that there is a wide tolerance in aligning the patch 100 and base unit 200. In this way, users can consistently connect patches 100 with the base unit 200 without the need for excessive care.

FIG. 11 is similar to FIG. 10 but shows an alternative arrangement. Instead of providing a thick layer 15 of harder material with the electrodes 42, 44, 46 extending directly out of the end of the sensor tube 22 into the harder layer 15, the electrodes 42, 44, 46 extend into the body 10 of the patch 100 and only their end portions are set in, and exposed at the upper surface of a thinner, relatively hard layer 16. It will be apparent from this that the electrodes 42, 44, 46 need not extend out of the top of the tube 22 or other sensor body 20 but can extend out of the sides.

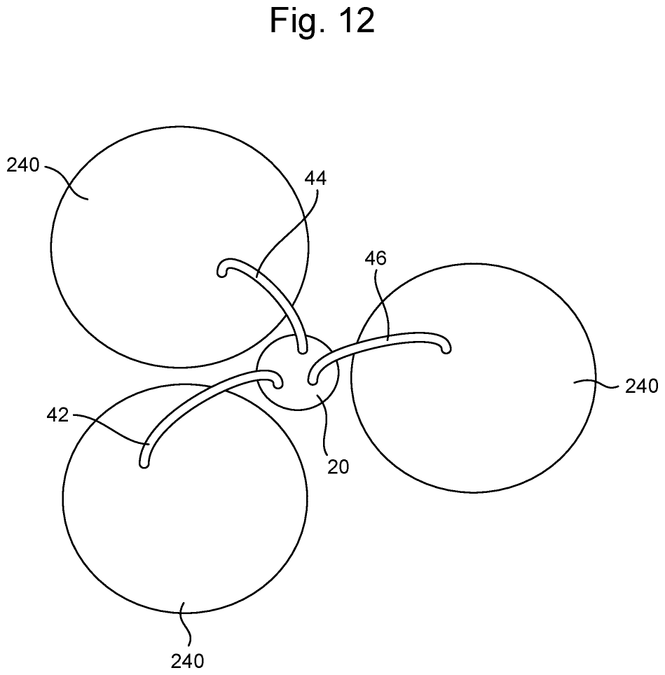

FIG. 12 is a schematic view corresponding FIG. 9, showing alignment of base unit pads 240 and electrodes 42, 44, 46 extending out of the sensor tube 22 of the patch in FIGS. 10 and 11. Although in these figures it appears that the electrodes 42, 44, 46 extend over the base unit pads 240, this is only for ease of drawing and it will be appreciated that in practice the electrodes 42, 44, 46 are arranged below the pads 240.

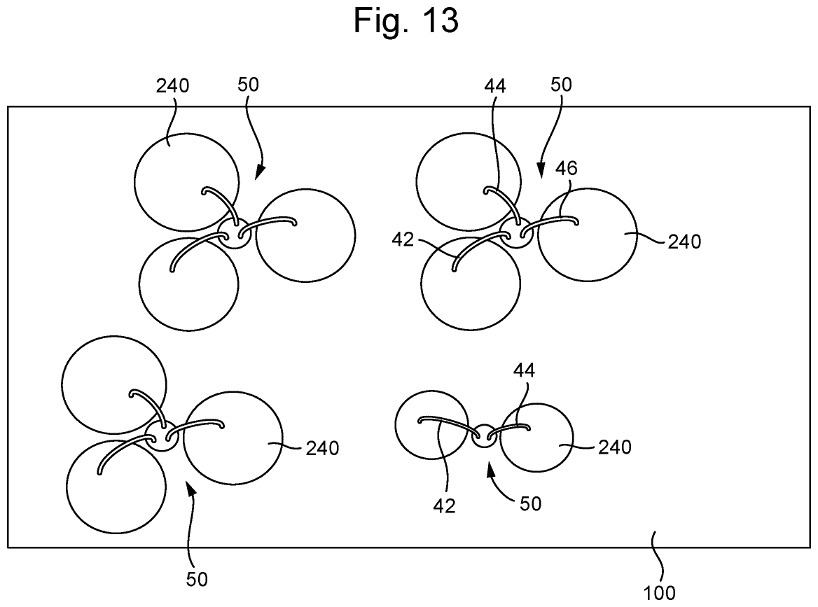

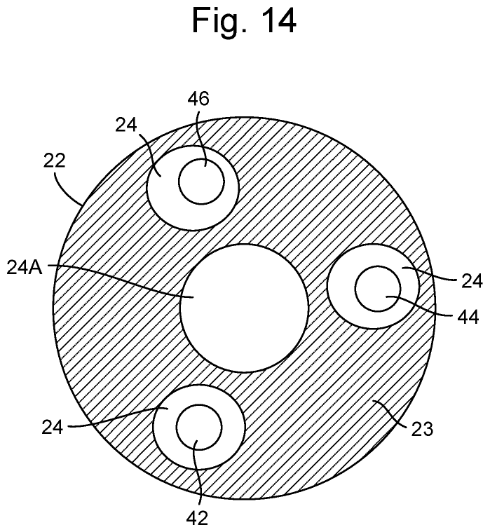

FIG. 13 shows a single patch 100 having a plurality of sensors 50, all connecting to pads 240 on the same base unit 200 (with other elements of the base unit 200 being omitted from the drawing). The sensors 50 may sense the same or different parameters, e.g. glucose, lactate, cortisol, sodium etc. It will be apparent that a similar arrangement could be provided using the type of connection shown in FIG. 8, or a mix of connection types between the electrodes 42, 44, 46 in the sensors 50 on the patch 100 and corresponding electrodes, such as pads 240, on the base unit 200 can be used.

A cross-section of an alternative embodiment of the sensor 50 is shown in FIG. 14. The sensor 50 in this embodiment may be used in place of the sensor 50 in any other embodiment. In this embodiment, the sensor body 20 comprises a tube 22 comprising a tube wall 23. A hole 24 is provided in the tube wall 23 for each electrode wire 42, 44, 46. In the illustrated example, the electrode wires consist of a working electrode 42 and a counter electrode 44 both formed of Pt and a reference electrode 46 formed of Ag, preferably coated with AgCl. However, as above, this is not limiting and fewer or more electrodes may be provided.

The tube 22 may be formed, for example, of PEEK having an external diameter of 360 μm. More preferably, the tube is Portex® PVC tubing having an external diameter of 800 μm. This has the advantage that the tube is easier to cut and handle. The holes 24 may be formed in the tubing using, for example, a femtosecond laser or by moulding when the tube is formed or by extrusion.

The electrode wires 42, 44, 46 may have a diameter of, for example, 20 μm (or 50 um). The internal diameter of the holes 24 may be, for example, 100 μm. However, any suitable dimensions may be chosen so long as the electrode wires can be disposed in the respective holes 24. The tube and wire diameters discussed in respect of the above embodiments may also be used.

The bead 30 may then be formed on the end of the tube 22 to cover substantially its whole diameter so that the tips of the electrode wires 42, 44, 46 are all covered and in contact with the sensing bead 30.

This arrangement has the advantage that the positions of the electrode wires 42, 44, 46 can be more easily controlled, which improves the accuracy and reproducibility of the sensor 50.

In addition, it is not necessary to include the process step of disposing the adhesive in the central bore 24A of the tube 22 with the electrode wires 42, 44, 46. This step may damage the electrode wires 42, 44, 46. The central bore 24A may be left empty or filled to increase the strength of the tube 22, although there may be a corresponding reduction in the flexibility of the tube.

Moreover, by disposing different electrode wires 42, 44, 46 in different holes 24, they can be electrically insulated from one another by the material of the tube body 23. Thus, it is not necessary to provide insulated wires. By contrast, where it is necessary to provide insulated wired, the insulation must be thin and is liable to damage during manufacturing of the sensor 50.

In a modification applicable to any embodiment, the sensor body 20 need not comprise a tube 22 and instead may be a solid, electrically non-conductive body with holes 24 formed in it for the respective electrode wires 42, 44, 46. For example, a solid rod with holes 24 formed in it may be used instead. The cross-section of the tube, rod or other body need not be circular and can be any appropriate shape.

In another embodiment, which is independent of but may be used in combination with any other embodiment described herein, in order to position accurately the sensor patch 100 with respect to the base unit 200, one side of the sensor 100a patch is provided with a specific 3D shape 110 (square, rectangular, round, oval, drop shape, polygon shape) protruding at a specific position. This specific shape is the negative of a corresponding concave shape 260a on the base unit side making the attachment easy. The electrodes 42, 44, 46 and the pads 240 are at another known position. One or more of the following points may apply:
The concave shape 260a can be provided in the base unit case 210 using a mold.
The added element 110 on the patch 100a could be provided using parylene with a top mold. Multiple protrusions 110 may be provided on the mould and multiple indentations 260a of corresponding shape may be provided on the base unit 200a.
The protrusion(s) 110 may be provided on the base unit 200a and the indentation(s) 260a on the patch 100a.
If multiple protrusions/indentations are provided, they may have different shapes.
If multiple protrusions/indentations are provided, their positions may not have rotational symmetry to ensure the correct alignment of the patch and the base unit.

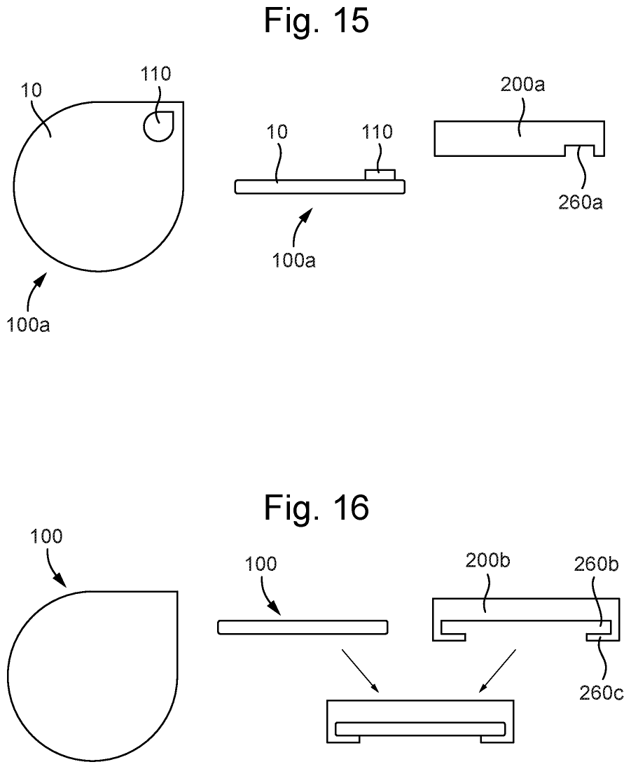

An exemplary arrangement is shown in FIG. 15.

In another embodiment, which is independent of but may be used in combination with any other embodiment described herein, as shown in FIG. 16, the base unit 200b may form or include one or more grooves 260b that the patch 100 is clipped in.

The groove 260b may be formed by a leaf 260c and the back surface of the base unit 200b. The leaf 260c may be sprung or hinged, for example using a living hinge, or may be substantially rigid. In FIG. 16, the grooves 260b are shown as open before the patch 100 is clipped in. However, the leaf 260c forming the groove 260b may contact the back surface of the base unit 200b before the patch 100 is inserted. The leaf 260b is then moved away from the back surface of the base unit 200b to insert the patch 100, before it springs back into place to clamp the patch. Any suitable clamping mechanism may be used. Thus, the base unit 200b may include clamps, which clamp onto the side edges of the patch 100. For example, the leaf 260c may be formed as a separate component to the base unit 200b attached with a hinge. A spring (not shown) may be provided to bias the leaf 260c towards the back surface of the base unit 200b.

It is not necessary for the base unit 200b to hold the patch 100 by the side edges. For example, the patch 100 may be provided with a projection (not shown) which slides into the grooves 260b or is clamped by a clamp on the base unit. It will be apparent that the patch 100 may include the grooves/clamps 260b for holding the base unit 200.

The base unit may also include a clamp for attachment of the sensor device 1 (combined patch 100 and base unit 200, 200a, 200b) to an item of clothing.

If the patch 100 includes a layer 15, 16 to provide a degree of resilience/rigidity, the patch 100 can be bent so its sides or portions acting as tongues fit into grooves 260b formed in the base unit 200b and then released to return to its natural shape. The patch 100 is then held in place by the tongues in grooves 260b. Alternatively, if sufficiently rigid, the patch 100 can simply be slid into holding grooves 260b provided on the base unit 200b.

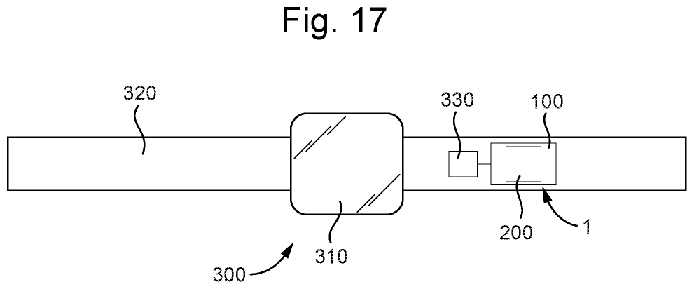

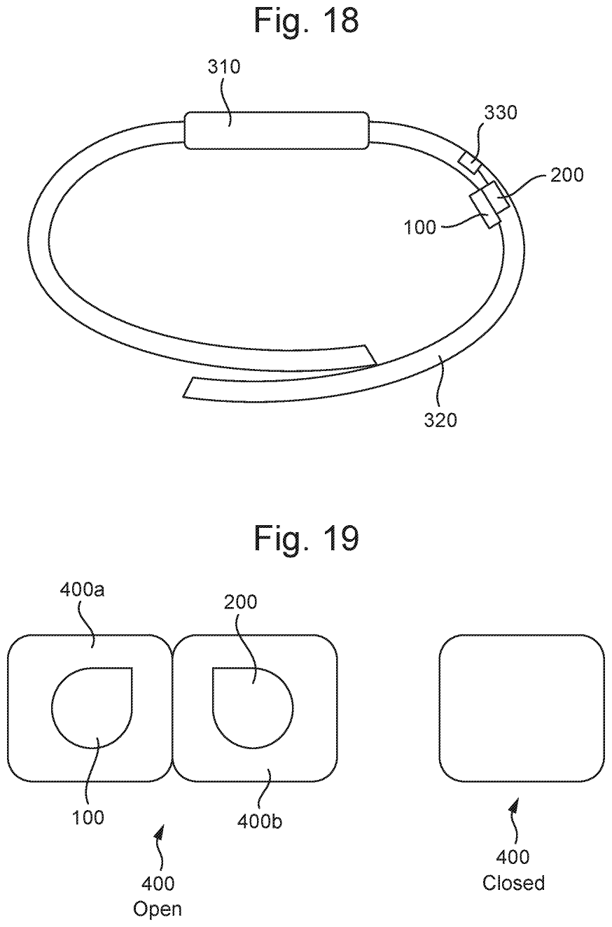

In another embodiment, which is independent of but may be used in combination with any other embodiment described herein, as shown in FIGS. 17 and 18, the sensor device 1 (electronics and patch) may be provided as a modular element for incorporation into, integration with, addition to or use with another device 300. For example, the sensor device 1 (sensor patch 100 and electronics 200) may be provided in a wrist band/strap 320 for use with a smart watch 300. The wrist band 300 with the electronics 200 can be sold with or separately from the smart watch 320 or other device, and disposable patches 100 can also be sold separately or together with the smart device 300 and/or wrist band 320. The electronics 200 may include means for communicating with the smart watch 300 or other device, for example a BLE receiver and transmitter, or wired means.

A battery 330, which is preferably rechargeable or replaceable, may also be provided in the strap 320. Rather than providing a battery 330 in the wrist band 320, the electronics 200 may be powered by the battery (not shown) of the smart watch 300 or other device.

The patch 100 and the electronics 200 may be integrally provided, but it is preferred that the electronics (base unit) 200 are integrated in the body of the strap 320 (or provided protruding to the outside of the strap or in a band around the strap) and the consumable patch 100 is removably provided on the inside of the strap 320 adjacent the wearer's skin 5.

The use of a wrist band 320 provides the right amount of pressure of the sensor patch 100 on the skin 5. If the patch 100 is pressed too hard, the sensitivity of the sensor bead 30 is reduced. Conversely if the patch 100 is not pressed hard enough, the sensor bead 30 may not be sufficiently in contact with or close enough to the skin 5. However, the right amount of pressure can be obtained with a wrist band 320, particularly when a wrist band with a snap close such as that shown in FIG. 18 is provided.

The electronics 200 may be provided in the main body of the smart watch 300 or other device itself and the electrodes 42, 44, 46 from the patch 100 may attach to carrier wires in the strap 320, which are in turn connected to the electronics.

The device 300 need not be a smart watch but can be any other device, such as a fitness device.

The electronics 200, battery 320 and patch 100 may be provided in a wrist band independently of another device to create a smart wrist band. The smart wrist band can be used alone without any other device. Alternatively, the smart wrist band can be attached to another device such as a smart watch, fitness device, analogue watch, and so forth, but used independently of it.

If no other device is provided on the band, the smart band can connect with an external device such as a mobile phone or laptop by wires connected to sockets in the smart band or more preferably by BLE, other Bluetooth connection or Wi-Fi. The smart band may also communicate with such external devices even if another device such as a smart watch or fitness device is provided on or attached to the wrist band, also even if the smart band communicates with that other device.

In another embodiment, which is independent of but may be used in combination with any other embodiment described herein, the base unit 200 may sample the sensor(s) of the patch 100 with a first frequency, convert the results into a digital format using an A/D converter, and store the results in an onboard memory (not shown). The base unit may then communicate with an external device, such as a smartphone, to upload the stored results of sampling (whether fully or partly processed) at a different frequency or in bursts with a different timing.

For example, the base unit 200 may only upload the sampling results to the external device once every 5 minutes, or only when called to do so by the external device. For example, the user may go for a run wearing the sensing device 1 (patch 100 and base unit 200 together), the sensing device connected to the user's smart watch by Bluetooth. The base unit 200 may upload the results to the smart watch at a predetermined schedule, or when the amount of data stored in the memory of the base unit 200 reaches a threshold, or when the user looks at the smart watch to check his or her glucose levels.

In another alternative, the sensor device 1 may collect sensor data in intermittent bursts to gather reliable data. For example, the sensor device 1 may collect a plurality of measurements every few minutes (for instance, every 1-5 minutes), where each burst contains a plurality of sensor measurements. The duration or length of the measurement bursts may be determined by at least one of a duration of a burst (e.g., 10-60 seconds), number of measurements in a burst (e.g., 10-100), and measurement frequency of a burst (e.g., 1-50 Hz). Any of the parameters of the bursts may be varied depending on the system requirements or context. The parameters may be adjusted according to resource requirements, for example to control the battery life of the sensor system.

In any of the embodiments mentioned above, the sensor data may be processed through one or more processing methods to improve sensor data quality and/or data reliability. For example, to improve the signal to noise ratio a digital smoothing polynomial filter or least-squares smoothing filter such as Savitzky-Golay filter can be used. When such a digital filter is applied to a set of digital data points, it may increase the precision of the data without distorting the signal tendency. Other types of filters such as Kalman filters or outlier filters may be used to improve the sensor measurements. Such an arrangement significantly reduces the power requirements on the sensing device 1, meaning it can be used for much longer without charging, without affecting the user experience.

Data may be uploaded to an external device such as a server via the cloud/a gateway. Upload may be direct from the base unit 200 or via an intermediate device such as a smart watch or smart phone. The data may be subjected to Al or other analysis to obtain insights into the user's health and performance, which can subsequently be shared with the user.

The data from the sensing device 1 may be used in combination with data from other exercise tracking devices (such as a heart rate monitor, step counter, GPS tracker etc), with the data from different devices being married using a time signature.

In another embodiment, which is independent of but may be used in combination with any other embodiment described herein, as shown in FIG. 19, the sensor device 1 (both sensor patch 100 and base unit 200) is protected from the environment by a protective casing 400. As shown in FIG. 19, the casing 400 includes two hinged bodies 400a, 400b, each with an indentation. One side stores the base unit 200 and the other side stores one or more patches 100. The casing 400 will preferably combine at least three of the following functions, but not limited to three:
The casing 400 is charged from an outside port (not shown).
The casing 400 is used to charge the base unit 200.
The casing 400 is used to store the sensor device 1, protecting it from the environment.
The casing 400 is used to store multiple biosensor patches 100 in a protected envelope (for instance four patches 100, each patch 100 being stored for instance in a controlled environment, for example in a vacuum within a protected pouch).
The casing 400 may have means for communicating with an external computing device (mobile phone, laptop etc).
The casing 400 includes a memory element, which is used to store data. This is useful when the user is off grid and the data cannot be sent to the cloud/gateway/server. This is used as a temporary storage. It allows a user to collect relatively large volumes of data across many hours or days, without needing to connect to a smart watch or the like, and without requiring the memory in the base unit 200 to be too large. The data stored in the casing can be uploaded to the cloud/gateway/server when a connection is next available.
The casing 400 is used to position the sensor patch 100 correctly onto the base unit 200. In this concept, the base unit 200 is placed in the indentation on one side 400a and the patch 100 is placed in the indentation on the other side 400b. The act of closing the casing 400 combines the patch 100 and base unit 200 with the correct alignment, for example (but not necessarily) using one of the solutions discussed above.
The casing 400 has a defined place for the base unit 200, including metallic and magnetic pins (not shown) for charging the base unit 200.
The casing 400 has a defined place for the sensor patch 100 allowing the sensor patch 100 to be clipped in place.
The casing 400 can store the base unit 200 and sensor patch 100 when combined.

Embodiments of the present invention have been described by way of example only. It will be appreciated by those skilled in the art that modifications may be made to any one or more of the embodiments and that they may be used separately or in any suitable combination without departing from the scope of the invention as defined by the appended claims.

1. A sensor for measuring a biometric parameter of a bodily fluid, the sensor comprising:
a sensor body having first and second ends;
at least one electrode extending in the sensor body, whereby an end of the at least one electrode is exposed at a first end of the sensor body;
a sensor bead disposed at the first end of the sensor body and in contact with the exposed end of the at least one electrode.

2. The sensor according to claim 1, wherein the sensor bead is hydrophilic and is configured to equilibrate with the bodily fluid by molecular or ionic exchange with the bodily fluid; and
the at least one electrode is configured to obtain a signal relating to the concentration of the hydrophilic sensor bead relating to the parameter.

3. (canceled)

4. The sensor according to claim 1, wherein the sensor body comprises one of a tube and a conduit.

5. The sensor according to claim 4, wherein a hole is formed in a wall of the one of the tube and the conduit, said hole extending in an axial direction of the one of the tube and the conduit and the at least one electrode is disposed in the hole.

6. The sensor according to claim 5, comprising a plurality of electrodes and a plurality of holes in the wall of the sensor body, each electrode being disposed in a respective hole, whereby the electrodes are insulated from one another.

7. The sensor according to claim 4, wherein the sensor body comprises a material, for example a cured adhesive, in which the at least one electrode is embedded.

8. The sensor according to claim 1, wherein the sensor bead comprises a hydrophilic hydrogel encapsulating an enzyme.

9. (canceled)

10. (canceled)

11. The sensor according to claim 1, wherein the sensor bead is coated with a diffusion limiting layer.

12. (canceled)

13. The sensor according to claim 1, wherein the at least one electrode comprises a working electrode, and an exclusion layer is provided on the working electrode at the interface between the working electrode and the sensor bead.

14. (canceled)

15. (canceled)

16. (canceled)

17. (canceled)

18. (canceled)

19. A sensor patch for measuring a biometric parameter of a bodily fluid, the patch comprising:
a patch body;
a sensor bead; and
at least one electrode in contact with sensor bead;
wherein the sensor bead is exposed on a first side of the patch for contacting a bodily fluid of a user.

20. The sensor patch according to claim 19, further comprising a sensor body disposed in or on the patch body, the sensor bead being disposed at one of the sensor body the at least one electrode being disposed in the sensor body.

21. (canceled)

22. (canceled)

23. A sensor patch for measuring a biometric parameter of a bodily fluid, the patch comprising:
a patch body; and
the sensor according to claim 1,wherein the sensor bead is exposed on a first side of the patch for contacting a bodily fluid of a user.

24. The sensor patch according to claim 23, wherein the sensor body extends to a second side of the patch body opposite the first side and the at least one electrode is exposed on the second side.

25. The sensor patch according to claim 23, further comprising a layer of polymer material on a second side of the patch body opposite to the first side, wherein the polymer material is harder than the material of the patch body, and the at least one electrode extends through the layer of polymer material.

26. (canceled)

27. The sensor patch according to claim 23, wherein the sensor bead protrudes from the surface of the patch on the first side whereby it can be pressed in contact with the user's skin when the patch is worn

28. The sensor patch according to claim 23, wherein the sensor bead is at least partly recessed in the patch.

29. (canceled)

30. (canceled)

31. A sensor device comprising:
a base unit; and
the sensor patch according to claim 23,
wherein the sensor patch is removably attachable to the base unit, and
the base unit comprises electronics for processing signals received from the at least one electrode.

32. (canceled)

33. A method of making a sensor, comprising:
disposing at least one electrode in a sensor body;
depositing a sensor bead on the end of the sensor body in contact with the at least one electrode.

34. A method according to claim 33, wherein the sensor body comprises a tube, the method comprising:
introducing an adhesive into the tube surrounding the at least one electrode;
curing the adhesive;
removing an end of the tube to expose an end of the at least one electrode.

35. A method according to claim 33, wherein disposing at least one electrode in a sensor body comprises disposing the at least one electrode to extend in a wall of the sensor body in an axial direction of the sensor body.

36. (canceled)

37. (canceled)

38. (canceled)
